(19) United States
(12) Patent Application Publication
(10) Pub. No.: US 2025/0025681 A1
(43) Pub. Date: Jan. 23, 2025

(54) ENERGY HARVESTING FOR CREATING CIRCULATION IN LEFT ATRIAL APPENDAGE

(71) Applicant: BOSTON SCIENTIFIC SCIMED, INC., MAPLE GROVE, MN (US)

(72) Inventors: Jerry Wang, Edina, MN (US); Joshua Mark Inouye, Brooklyn Park, MN (US); James M. Anderson, Corcoran, MN (US)

(73) Assignee: BOSTON SCIENTIFIC SCIMED, INC., MAPLE GROVE, MN (US)

(21) Appl. No.: 18/775,516

(22) Filed: Jul. 17, 2024

Related U.S. Application Data
(60) Provisional application No. 63/527,370, filed on Jul. 18, 2023.

(51) Int. Cl. A61M 60/882; A61M 60/187; A61M 60/289; A61M 60/47; A61M 60/492; A61M 60/839; A61M 60/861

(52) U.S. Cl. CPC A61M 60/882; A61M 60/187; A61M 60/289; A61M 60/47; A61M 60/492; A61M 60/839; A61M 60/861; A61M 2205/8281

(57) ABSTRACT

A medical device for causing blood flow within a left atrial appendage (LAA) includes a flow energy capture element adapted to capture energy caused by movement within the heart. A transmission element is operably coupled to the flow energy capture element, the transmission element adapted to utilize the captured energy to cause blood flow within the LAA.

### CROSS REFERENCE TO RELATED APPLICATIONS

This application claims the benefit of priority of U.S. Provisional Application No. 63/527,370 filed Jul. 18, 2023, the entire disclosure of which is hereby incorporated by reference.

### TECHNICAL FIELD

The disclosure relates generally to medical devices and more particularly to medical devices that are adapted for preventing blood stagnation within a left atrial appendage.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a medical device for causing blood flow within a left atrial appendage (LAA). The medical device includes a flow energy capture element adapted to capture energy caused by movement within the heart and a transmission element operably coupled to the flow energy capture element, the transmission element adapted to utilize the captured energy to cause blood flow within the LAA.

Alternatively or additionally, the flow energy capture element includes a first moment arm and the transmission element includes a second moment arm operably coupled with the first moment arm.

Alternatively or additionally, the medical device may further include an attachment point disposed between the first moment arm and the second moment arm, the attachment point adapted to be secured to cardiac tissue between the LAA and a mitral valve, thereby positioning the first moment arm within blood flow caused by blood flowing through the mitral valve and positioning the second moment arm within the LAA.

Alternatively or additionally, the medical device may further include a biasing element adapted to move the first moment arm against blood flow.

Alternatively or additionally, the biasing element may include a spring.

Alternatively or additionally, the biasing element may include one or more magnets.

Alternatively or additionally, the flow energy capture element may include a sail element adapted to move in response to impinging blood flow, and the transmission element may include a first tether having a first end secured to the sail element and a second end adapted to be secured to within a distal region of the LAA such that movement of the sail element causes the first tether to pull on the distal region of the LAA, thereby causing blood flow within the LAA.

Alternatively or additionally, the sail element may include a plurality of surfaces against which blood may impinge.

Alternatively or additionally, the medical device may further include a second tether having a first end secured to the sail element and a second end adapted to be secured to a septal wall.

Alternatively or additionally, the flow energy capture device may include an impeller adapted to rotate in response to blood flow past the impeller.

Alternatively or additionally, the impeller may include a helical impeller adapted to rotate in response to impinging blood.

Alternatively or additionally, the transmission element may include a windup tether including a first end secured to the impeller and a second end adapted to be secured within a distal region of the LAA such that rotation of the impeller causes the windup tether to wind up and unwind, thereby pulling on the distal region of the LAA, thereby causing blood flow within the LAA.

Alternatively or additionally, the transmission element may include a tether having a first end and a second end, the first end secured to the impeller, and a propeller operably coupled to the second end, the propeller adapted to be disposed within the LAA such that rotation of the impeller causes rotation of the tether, which in turn causes rotation of the propeller in order to cause blood flow within the LAA. The tether may be adapted to be secured relative to an ostium of the LAA.

Alternatively or additionally, the flow energy capture element may include a parachute adapted to be deployed within a left ventricle, the parachute adapted to move in response to impinging blood flow, and the transmission element may include a tether having a first end secured to the parachute and a second end adapted to be secured within a distal region of the LAA such that the tether pulls on the distal region of the LAA in response to movement of the parachute, thereby causing blood flow within the LAA.

Alternatively or additionally, the tether may be adapted to pass through a mitral valve.

Another example may be found in a medical device for causing blood flow within a left atrial appendage (LAA). The medical device includes a flow energy capture element adapted to move in response to blood flow within the heart, and a transmission element including a first end operably coupled to the flow energy capture element and a second end adapted to be secured to a distal region of the LAA such that movement of the flow energy capture element causes the transmission element to exert a force on the distal region of the LAA.

Alternatively or additionally, the flow energy capture element may include a sail.

Alternatively or additionally, the flow energy capture element may include a parachute.

Alternatively or additionally, the flow energy capture element may include an impeller.

Another example may be found in a medical device for causing blood flow within a left atrial appendage (LAA). The medical device includes a flow energy capture element adapted to move in response to blood flowing from the left atrium to the left ventricle, and a transmission element including a first end operably coupled to the flow energy capture element and a second end adapted to be secured to a distal region of the LAA.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic illustration of a human heart;

FIG. 2 is a partial cross-sectional view of an LAA (left atrial appendage);

FIG. 3 is a schematic view of an illustrative medical device for causing blood flow within the LAA shown disposed within the heart;

FIG. 4 is a schematic view of an illustrative medical device for causing blood flow within the LAA shown disposed within the heart;

FIG. 4A is a schematic view of the illustrative medical device of FIG. 4, including a mechanical biasing structure;

FIG. 4B is a schematic view of the illustrative medical device of FIG. 4, including a magnetic biasing structure;

FIG. 5 is a schematic view of an illustrative medical device for causing blood flow within the LAA shown disposed within the heart;

FIG. 5A is a cross-sectional view taken along the line 5A-5A of FIG. 5;

FIG. 6 is a schematic view of a portion of the LAA, showing how the illustrative medical device of FIG. 5 impacts the heart;

FIG. 7 is a schematic view of an illustrative medical device for causing blood flow within the LAA shown disposed within the heart;

FIG. 8 is a schematic view of an illustrative medical device for causing blood flow within the LAA shown disposed within the heart;

FIG. 9 is a schematic view of an illustrative medical device for causing blood flow within the LAA shown disposed within the heart at a first stage in the cardiac cycle; and

FIG. 10 is a schematic view of an illustrative medical device for causing blood flow within the LAA shown disposed within the heart at a second stage in the cardiac cycle.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

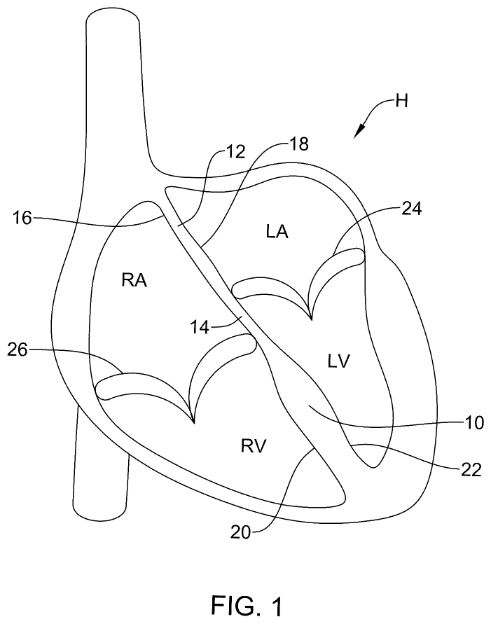

FIG. 1 is a schematic illustration of a human heart H. The heart H includes a right side and a left side, relative to the person's perspective. The right side of the heart H includes an RA (right atrium) and an RV (right ventricle). The left side of the heart H includes an LA (left atrium) and an LV (left ventricle). A ventricular septum 10 separates the RV and the LV and an atrial septum 12 separates RA and the LA. The heart H may also be considered as including an atrioventricular septum 14 between the RA and the LV. The ventricular septum 10 may be considered as having an RV facing side 16 and an LV facing side 18. The atrial septum 11 may be considered as including a RA facing side 20 and a RA facing side 22. A mitral valve 24 is disposed between the LA and the LV, and controls blood flow from the LA into the LV. A tricuspid valve 26 is disposed between the RA and the RV, and controls blood flow from the RA into the RV.

It will be appreciated that this view of the heart H is highly schematic, and shows a slice through the heart H that only shows the mitral valve 24 and the tricuspid valve. The pulmonary valve, which controls blood flow from the RV to the patient's lungs, and the aortic valve, which controls blood from to the body from the LV, are not shown in this view. Also not shown in this schematic slide is the left atrial appendage, or LAA. The LAA is an appendage that may be considered as being part of the LA.

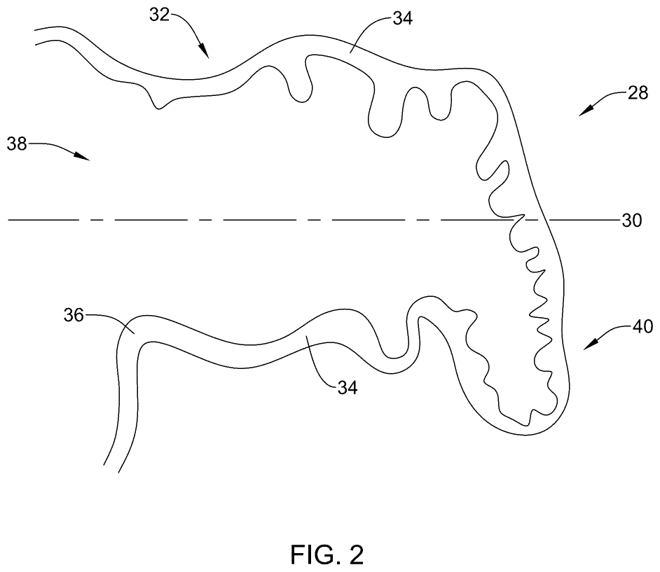

FIG. 2 is a partial cross-sectional view of a left atrial appendage 28. In some embodiments, the left atrial appendage (LAA) 28 may have a complex geometry and/or irregular surface area. It will be appreciated that the illustrated LAA 28 is merely one of many possible shapes and sizes for the LAA 28, which may vary from patient to patient. Those of skill in the art will also recognize that the medical devices, systems, and/or methods disclosed herein may be adapted for various sizes and shapes of the LAA 28, as necessary. The left atrial appendage 28 may include a generally longitudinal axis 30 arranged along a depth of a main body 32 of the left atrial appendage 28. The main body 32 may include a lateral wall 34 and an ostium 36 forming a proximal mouth 38. In some examples, a lateral extent of the ostium 36 and/or the lateral wall 34 may be smaller or less than a depth of the main body 32 along the longitudinal axis 30, or a depth of the main body 32 may be greater than a lateral extent of the ostium 36 and/or the lateral wall 24. In some examples, the LAA 28 may narrow quickly along the depth of the main body 32 or the left atrial appendage may maintain a generally constant lateral extent along a majority of depth of the main body 32. In some examples, the LAA 28 may include a distal region 40. In some instances, the distal region 40 may include a region formed or arranged as a tail-like element associated with a distal portion of the main body 32. In some examples, the distalmost region may protrude radially or laterally away from the longitudinal axis 30.

In some cases, and particularly during times of atrial fibrillation, blood flow within the LAA 10 may become stagnant. Stagnant blood flow within the LAA 28 may lead to blood clots, which can lead to stroke. In some cases, creating blood flow within the LAA 28 may help to reduce stagnation and thus reduce the likelihood of blood clot formation, especially during atrial fibrillation. FIGS. 2 through 10 provide examples of medical devices that may be implanted in or near the LAA 28 in order to reduce stagnation. In some instances, these medical devices may operate at all times. In some instances, these medical devices may be at least partially driven by blood moving within the heart. In some instances, these medical devices may be at least partially driven by movement of the heart itself, as the heart beats. It will be appreciated that the heart twists and squeezes with each heartbeat.

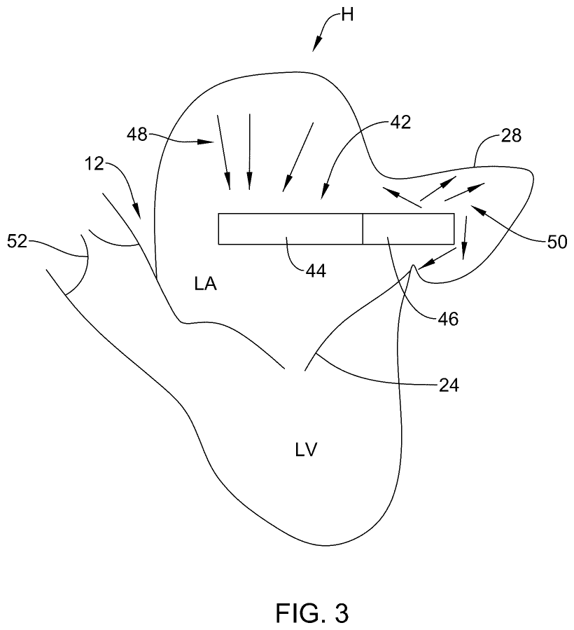

FIG. 3 is a schematic view of an illustrative medical device 42 that may be deployed within the heart H in order to cause blood flow within the LAA 28. In some instances, this can reduce the chances of blood stagnation causing blood clots to form within the LAA 28, particularly since blood clots in the left side of the heart H can travel to the patient's brain, with potentially catastrophic results. The medical device 42, which is shown schematically, includes a flow energy capture element 44 that is adapted to be positioned within the LA and a transmission element 46 that is adapted to be positioned at least partially within the LAA 28. In some instances, the medical device 42 or components thereof may be formed of shape memory alloys and polymers. In some instances, the medical device 42 or components thereof may be formed of stainless steel and stainless steel alloys. In some instances, at least portions of the medical device 42 may include radiopaque elements.

In some instances, the flow energy capture element 44 may be considered as functioning as a first moment arm and the transmission element 46 may be considered as functioning as a second moment arm. In some instances, relative length differences between the first moment arm and the second moment arm may provide a mechanical advantage through force multiplication. In some instances, this may mean that the medical device 42 may be made smaller. In some instances, the flow energy capture element 44 may be counter-balanced in order to better self-align the flow energy capture element 44 normal to blood flow in order to maximize flow energy harvesting. In some instances, without counter-balancing, the flow energy capture element 44 may simply freely spin without transferring energy to the transmission element 46. In some instances, without counter-balancing or other considerations, the flow energy capture element 44 may simply find an equilibrium position within the blood flow, and may stop moving. As blood flows within the LA, as indicated by arrows 48, the blood flow will cause the flow energy capture element 44 to start moving. As the flow energy capture element 44 starts to move, the transmission element 46 is driven into motion. In some instances, the flow energy capture element 44 may be considered as being adapted to capture some of the kinetic energy of the moving blood by itself beginning to move. This kinetic energy may be transferred to the transmission element 46, and then transferred into the blood within the LAA 28 as movement of the transmission element 46 causes blood within the LAA 28 to move.

As the transmission element 46 starts moving, this movement causes blood within the LAA 28 to start moving, as indicated by arrows 50. It will be appreciated that this particular slice through the heart H also shows an aortic valve 52. In some instances, the medical device 42 may include additional features and elements not shown, such as but not limited to one or more anchoring features that allow the medical device 42 to be secured in place at an appropriate location, with the flow energy capture element 44 positioned to be exposed to blood moving within the LA, and the transmission element 46 positioned such that movement of the transmission element 46 will cause blood to move within the LAA 28.

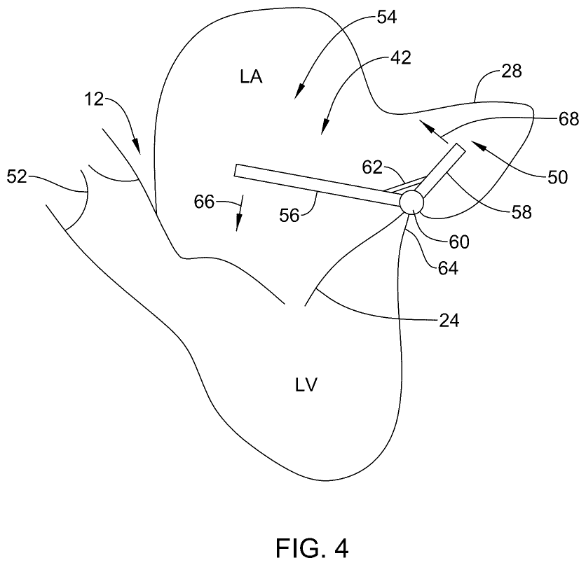

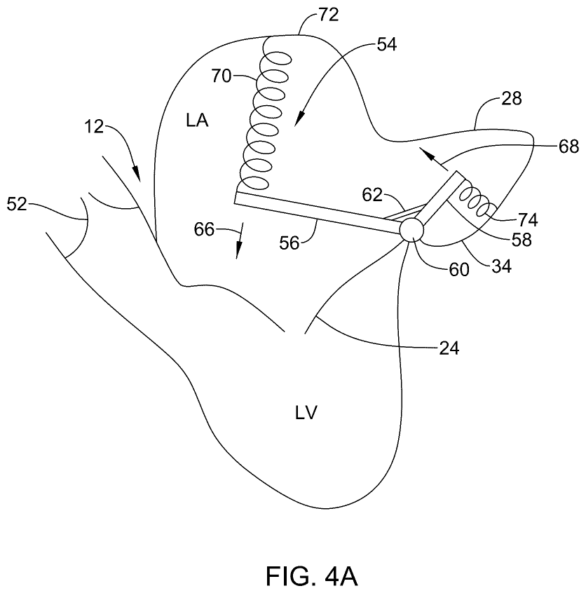

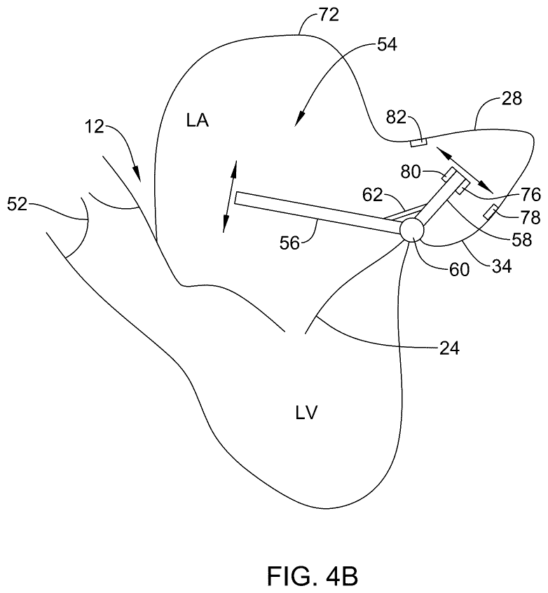

FIG. 4 is a schematic view of an illustrative medical device 54 that may be deployed within the heart H in order to cause blood flow within the LAA 28. In some instances, this can reduce the chances of blood stagnation causing blood clots to form within the LAA 28. The medical device 54, which is shown schematically, includes a flow energy capture element 56 that is adapted to be positioned within the LA and a transmission element 58 that is adapted to be positioned at least partially within the LAA 28. The medical device 54 includes a hinge or pivot point 60 that secures the flow energy capture element 56 relative to the transmission element 58. In some instances, the medical device 54 may also include a reinforcing member 62 that helps to secure the flow energy capture element 56 to the transmission element 58. The hinge or pivot point 60 may be adapted to be secured to tissue within the heart H. As an example, the hinge or pivot point 60 may be adapted to be secured to fibrous tissue within a mitral annulus 64.

In some instances, blood flowing from the LA into the LV through the mitral valve 24 may cause an end of the flow energy capture element 56 to move downward (in the illustrated orientation) in a direction indicated by an arrow 66. In response, an end of the transmission element 58 may be caused to move upward (in the illustrated orientation) in a direction indicated by an arrow 68. In some instances, the flow energy capture element 56 may move back in a direction opposite that indicated by the arrow 66 after the blood within the LA has passed through the mitral valve 24 and before the LA fills with blood again. In some instances, the medical device 54 may include a biasing structure that biases the medical device 54 against the blood flow.

In FIG. 4A, it can be seen that the medical device 54 now includes a mechanical biasing structure. As shown, the mechanical biasing structure may be a spring 70 that extends from the flow energy capture element 56 to an atrial wall 72. In some instances, the mechanical biasing structure may be a spring 74 that extends from the transmission element 58 to the lateral wall 34 of the LAA 28. In some instances, the medical device 54 may only include one of the spring 70 and the spring 74. In some instances, the medical device 54 may include both the spring 70 and the spring 74. In some instances, the hinge or pivot point 60 may itself include a spring other biasing element.

In FIG. 4B, it can be seen that the medical device 54 includes a magnetic biasing structure. As shown, the magnetic biasing structure may include a first magnet 76 that is secured to the transmission element 58 and a second magnet 78 that is adapted to be secured to the lateral wall 34 of the LAA 28. In some instances, the first magnet 76 and the second magnet 78 may be adapted to be attracted to each other. As an example, the first magnet 76 may present a North pole towards the second magnet 78 and the second magnet 78 may present a South pole towards the first magnet 76. Alternatively, the first magnet 76 may present a South pole towards the second magnet 78 and the second magnet 78 may present a North pole towards the first magnet 76. It will be appreciated that this attractive force will help to bias the flow energy capture element 56 against the blood flow.

In some instances, the magnetic biasing structure may include a first magnet 80 that is secured to the transmission element 58 and a second magnet 82 that is adapted to be secured to the lateral wall 34 of the LAA 28. In some instances, the first magnet 80 and the second magnet 82 may be adapted to repel to each other. As an example, the first magnet 80 may present a North pole towards the second magnet 82 and the second magnet 82 may present a North pole towards the first magnet 80. Alternatively, the first magnet 80 may present a South pole towards the second magnet 82 and the second magnet 82 may present a South pole towards the first magnet 80. It will be appreciated that this repellant force will help to bias the flow energy capture element 56 against the blood flow. While the magnetic biasing structure is shown as being disposed relative to the transmission element 58, it will be appreciated that similar magnetic biasing structures may also be disposed relative to the flow energy capture element 56, depending on relative distances for securing magnets to the tissue.

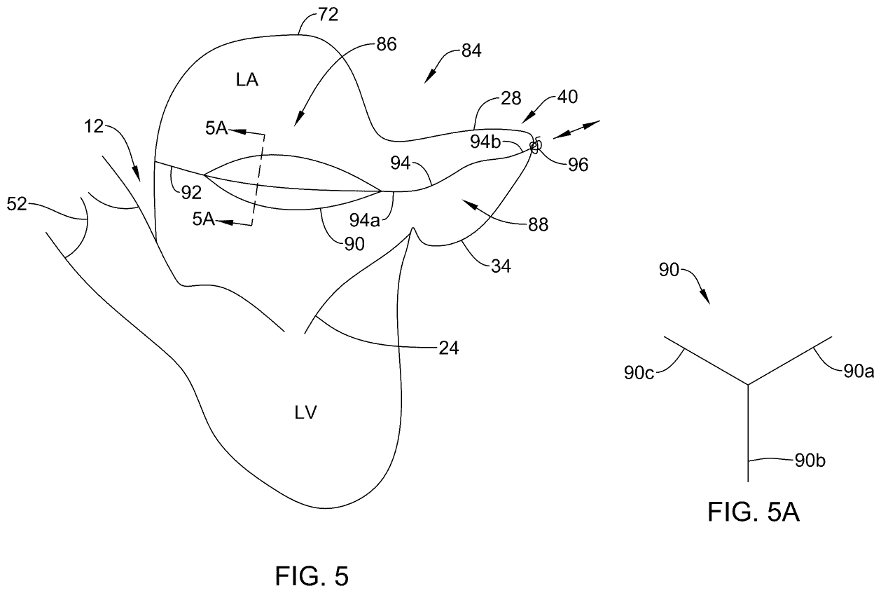

FIG. 5 is a schematic view of an illustrative medical device 84 that may be deployed within the heart H in order to cause blood flow within the LAA 28. In some instances, this can reduce the chances of blood stagnation causing blood clots to form within the LAA 28. The medical device 84, which is shown schematically, includes a flow energy capture element 86 that is adapted to be positioned within the LA and a transmission element 88 that is adapted to be positioned at least partially within the LAA 28. In some instances, the flow energy capture element 86 includes a sail element 90 and an anchor 92 that is adapted to be secured to the atrial septum 12. In some instances, the anchor 92 may be adapted to be secured to the atrioventricular septum 14 instead. In some instances, the transmission element 88 includes a tether 94 that includes a first end 94a that is coupled to the sail element 90 and a second end 94b that secured to an anchor 96 that is adapted to be secured to the distal region 40 of the LAA 28. In some instances, the anchor 96 may include a coil, suture or hook, for example.

The sail element 90 may include one, two, three or more panels that function as sails. The panels may be made of any suitable material that is thin and flexible, yet withstands the flow of blood within the LA. In some instances, the panels may be fabric or polymeric, for example. FIG. 5A is a cross-sectional view of the sail element 90, taken along the line 5A-5A in FIG. 5. As shown, the sail element 90 has a total of three panels, labeled as 90a, 90b and 90c. In some instances, the sail element 90 may only have one panel or two panels. In some instances, the sail element 90 may have four or more panels.

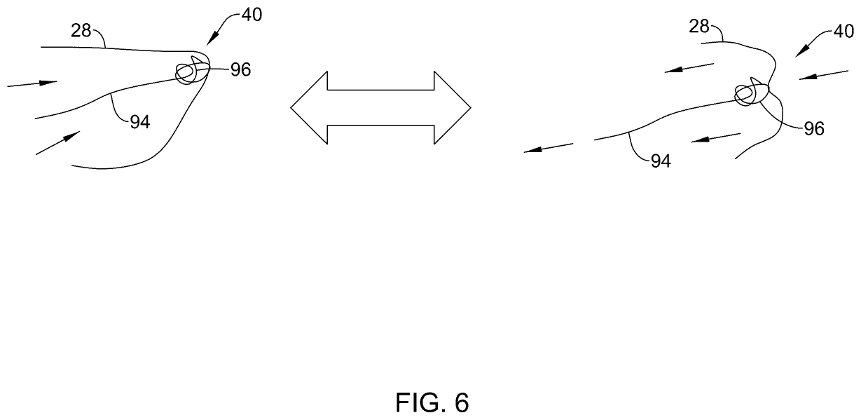

In use, the sail element 90 will move as blood impinges on the panels of the sail element 90. As the sail element 90 moves, it will pull on the tether 94. As seen in FIG. 6, when the sail element 90 is not moving as a result of blood movement within the LA, the LAA 28 retains its shape. This is seen on the left side of FIG. 6. However, when the sail element 90 is moving, and the tether 94 undergoes a resulting tensile force, the distal region 40 of the LAA 28 is pulled proximally. As the LAA 28 undergoes a temporary shape deformation, blood within the LAA 28 is caused to move. It will be appreciated that when blood flow within the LA ceases, at appropriate portions of the cardiac cycle, the LAA 28 will at least partially regain its original shape, and the process of periodically deforming the LAA 28 will continue as the cardiac cycle continues.

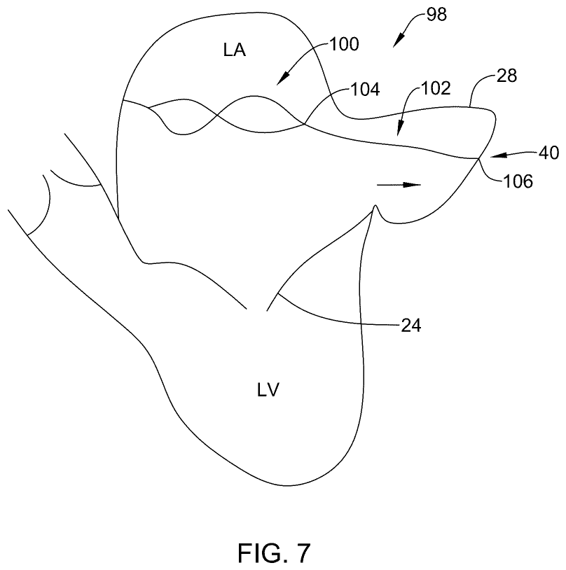

FIG. 7 is a schematic view of an illustrative medical device 98 that may be deployed within the heart H in order to cause blood flow within the LAA 28. In some instances, this can reduce the chances of blood stagnation causing blood clots to form within the LAA 28. The medical device 98, which is shown schematically, includes a flow energy capture element 100 that is adapted to be positioned within the LA and a transmission element 102 that is adapted to be positioned at least partially within the LAA 28. In some instances, the flow energy capture element 100 is an impeller. In some instances, the flow energy capture element 100 is a helical impeller. As blood impinges on the helical impeller, the helical impeller may be driven into rotation. As the helical impeller rotates, the transmission element 102 is caused to rotate in response. In some instances, the transmission element 102 is a windup tether. The windup tether includes a first end 104 that is secured to the impeller and a second end 106 that is adapted to be secured to the distal region 40 of the LAA 28. In some instances, rotation of the impeller causes the windup tether 102 to wind up and unwind, thereby pulling on the distal region 40 of the LAA 28, thereby causing blood flow within the LAA 28.

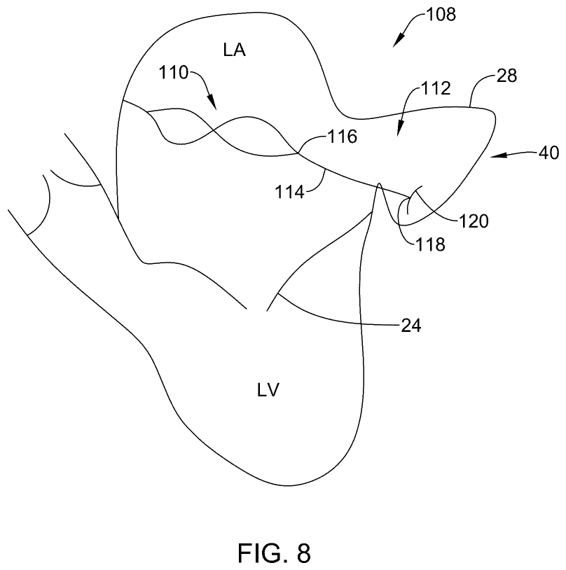

FIG. 8 is a schematic view of an illustrative medical device 108 that may be deployed within the heart H in order to cause blood flow within the LAA 28. In some instances, this can reduce the chances of blood stagnation causing blood clots to form within the LAA 28. The medical device 108, which is shown schematically, includes a flow energy capture element 110 that is adapted to be positioned within the LA and a transmission element 112 that is adapted to be positioned at least partially within the LAA 28. In some instances, the flow energy capture element 110 is an impeller. In some instances, the flow energy capture element 110 is a helical impeller. As blood impinges on the helical impeller, the helical impeller may be driven into rotation. As the helical impeller rotates, the transmission element 112 is caused to rotate in response. In some instances, the transmission element 112 includes a tether 114 having a first end 116 and a second end 118, the first end 116 secured to the impeller. The second end 118 is operably coupled with a propeller 120 that is adapted to be disposed within the LAA 28 such that rotation of the impeller causes rotation of the tether 114, which in turn causes rotation of the propeller 120 in order to cause blood flow within the LAA 28. In some instances, the tether 114 may be adapted to be secured relative to the ostium 36 of the LAA 28. In some instances, the tether 114 may extend through tissue proximate the LAA 28. The tether 114 may held in position by suturing or stapling into tissue proximate the LAA 28. In some instances, the tether 114 may be adhesively secured in place or with tissue adhering features on the tether 114 itself. In some instances, the tether 114 proximate the propeller 120 may extend through endoluminal tissue, the ostium of the LAA 28 or even the ventricular wall.

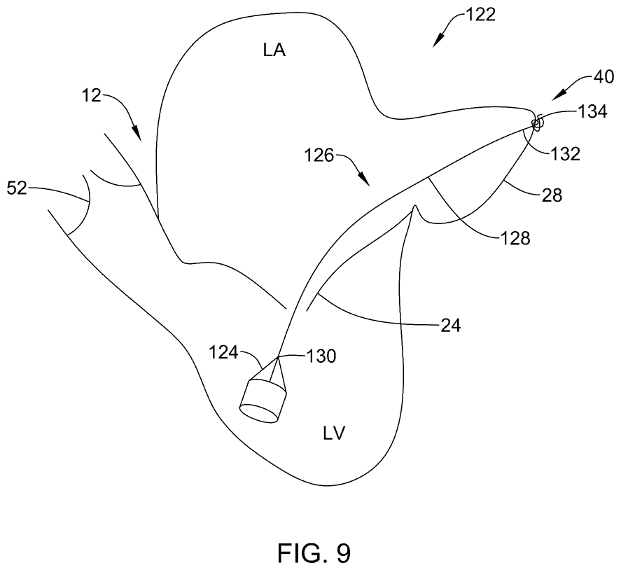

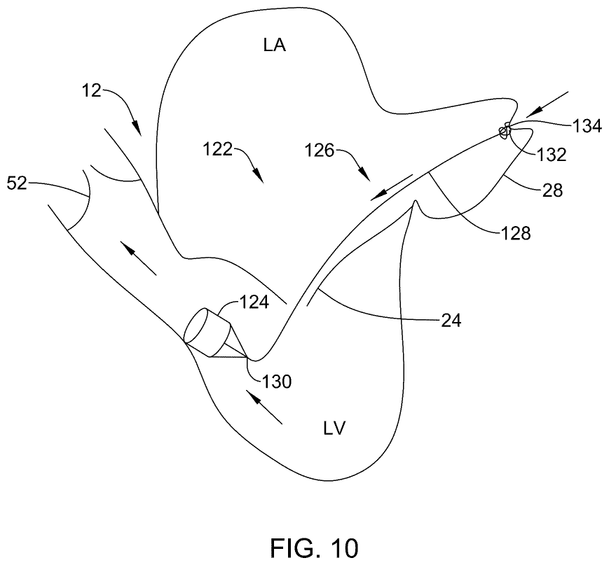

FIGS. 9 and 10 are schematic views of an illustrative medical device 122 that may be deployed within the heart H in order to cause blood flow within the LAA 28. In some instances, this can reduce the chances of blood stagnation causing blood clots to form within the LAA 28. FIG. 9 shows the medical device 122 during diastole while FIG. 10 shows the medical device during systole. In some instances, the medical device 122 includes a flow energy capture element 124 and a transmission element 126. In some instances the flow energy capture element is a parachute adapted to be deployed within the LV, the parachute adapted to move in response to impinging blood flow. In some instances, the transmission element 126 includes a tether 128 having a first end 130 that is secured to the parachute and a second end 132 that is adapted to be secured within the distal region 40 of the LAA 28, such as via an anchor 134, such that the tether 128 pulls on the distal region 40 of the LAA 28 in response to movement of the parachute, thereby causing blood flow within the LAA 28.

In comparing FIG. 9 and FIG. 10, it can be seen that in FIG. 10, blood flow from the LA to the LV, or possibly blood flow exiting the LV through the aortic valve 52, impinges on the parachute 124 and pulls the tether 128 further through the mitral valve 24. This causes the tether 128 to exert a tensile force on the distal region 40 of the LAA 28, thereby temporarily deforming the LAA 28. As the LAA 28 undergoes a temporary shape deformation, blood within the LAA 28 is caused to move. It will be appreciated that when blood flow within the LA ceases, at appropriate portions of the cardiac cycle, the LAA 28 will at least partially regain its original shape, and the process of periodically deforming the LAA 28 will continue as the cardiac cycle continues.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-clastic plateau” or “flag region” in its stress/strain curve like super-elastic nitinol does. Instead, in the linear-clastic and/or non-super-elastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear-elastic and/or non-super-elastic nitinol may also be termed “substantially” linear-elastic and/or non-super-clastic nitinol.

In some cases, linear-elastic and/or non-super-elastic nitinol may also be distinguishable from super-elastic nitinol in that linear-elastic and/or non-super-clastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear-clastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear-elastic and/or non-super-clastic nickel-titanium alloy maintains its linear-clastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for causing blood flow within a left atrial appendage (LAA), the medical device comprising:
a flow energy capture element adapted to capture energy caused by movement within the heart; and
a transmission element operably coupled to the flow energy capture element, the transmission element adapted to utilize the captured energy to cause blood flow within the LAA.

2. The medical device of claim 1, wherein:
the flow energy capture element comprises a first moment arm; and
the transmission element comprises a second moment arm operably coupled with the first moment arm.

3. The medical device of claim 2, further comprising an attachment point disposed between the first moment arm and the second moment arm, the attachment point adapted to be secured to cardiac tissue between the LAA and a mitral valve, thereby positioning the first moment arm within blood flow caused by blood flowing through the mitral valve and positioning the second moment arm within the LAA.

4. The medical device of claim 3, further comprising a biasing element adapted to move the first moment arm against blood flow.

5. The medical device of claim 4, wherein the biasing element comprises a spring.

6. The medical device of claim 4, wherein the biasing element comprises one or more magnets.

7. The medical device of claim 1, wherein:
the flow energy capture element comprises a sail element adapted to move in response to impinging blood flow;
the transmission element comprises a first tether having a first end secured to the sail element and a second end adapted to be secured to within a distal region of the LAA such that movement of the sail element causes the first tether to pull on the distal region of the LAA, thereby causing blood flow within the LAA.

8. The medical device of claim 7, wherein the sail element comprises a plurality of surfaces against which blood may impinge.

9. The medical device of claim 7, further comprising a second tether having a first end secured to the sail element and a second end adapted to be secured to a septal wall.

10. The medical device of claim 1, wherein the flow energy capture device comprises an impeller adapted to rotate in response to blood flow past the impeller.

11. The medical device of claim 10, wherein the impeller comprises a helical impeller adapted to rotate in response to impinging blood.

12. The medical device of claim 10, wherein the transmission element comprises a windup tether including a first end secured to the impeller and a second end adapted to be secured within a distal region of the LAA such that rotation of the impeller causes the windup tether to wind up and unwind, thereby pulling on the distal region of the LAA, thereby causing blood flow within the LAA.

13. The medical device of claim 10, wherein the transmission element comprises:
a tether having a first end and a second end, the first end secured to the impeller; and
a propeller operably coupled to the second end, the propeller adapted to be disposed within the LAA such that rotation of the impeller causes rotation of the tether, which in turn causes rotation of the propeller in order to cause blood flow within the LAA;
wherein the tether is adapted to be secured relative to an ostium of the LAA.

14. The medical device of claim 1, wherein:
the flow energy capture element comprises a parachute adapted to be deployed within a left ventricle, the parachute adapted to move in response to impinging blood flow;
the transmission element comprises a tether having a first end secured to the parachute and a second end adapted to be secured within a distal region of the LAA such that the tether pulls on the distal region of the LAA in response to movement of the parachute, thereby causing blood flow within the LAA.

15. The medical device of claim 14, wherein the tether is adapted to pass through a mitral valve.

16. A medical device for causing blood flow within a left atrial appendage (LAA), the medical device comprising:
a flow energy capture element adapted to move in response to blood flow within the heart; and
a transmission element including a first end operably coupled to the flow energy capture element and a second end adapted to be secured to a distal region of the LAA such that movement of the flow energy capture element causes the transmission element to exert a force on the distal region of the LAA.

17. The medical device of claim 16, wherein the flow energy capture element comprises a sail.

18. The medical device of claim 16, wherein the flow energy capture element comprises a parachute.

19. The medical device of claim 16, wherein the flow energy capture element comprises an impeller.

20. A medical device for causing blood flow within a left atrial appendage (LAA), the medical device comprising:
a flow energy capture element adapted to move in response to blood flowing from the left atrium to the left ventricle; and
a transmission element including a first end operably coupled to the flow energy capture element and a second end adapted to be secured to a distal region of the LAA.
